(19) United States
(12) Patent Application Publication
(10) Pub. No.: US 2002/0006971 A1
(43) Pub. Date: Jan. 17, 2002

(54) Process for the removal of organic interfering components from an alpha-olefin crude product

(76) Inventor: Hans Becker, Munchen (DE)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/847,700

(22) Filed: May 2, 2001

(30) Foreign Application Priority Data
May 9, 2000 (DE) DE 100 22 466.0

(51) Int. Cl. C07C 27/34

(52) U.S. Cl. 518/724; 203/58

(57) ABSTRACT

The invention relates to a process for the removal organic interfering components from an alpha olefin crude product recovered in a Fischer-Tropsch synthesis plant by extractive distillation using a scrubbing agent containing N-Methyl-2-Pyrrolidon (NMP), wherein the NMP is loaded with the organic interfering components, is then regenerated and subsequently reused in the extractive distillation. The alpha olefin crude product contains water in addition to the organic interfering components, optionally to saturation point and the scrubbing agent is freed of water during regeneration to a maximum content of 1000 ppm by weight.

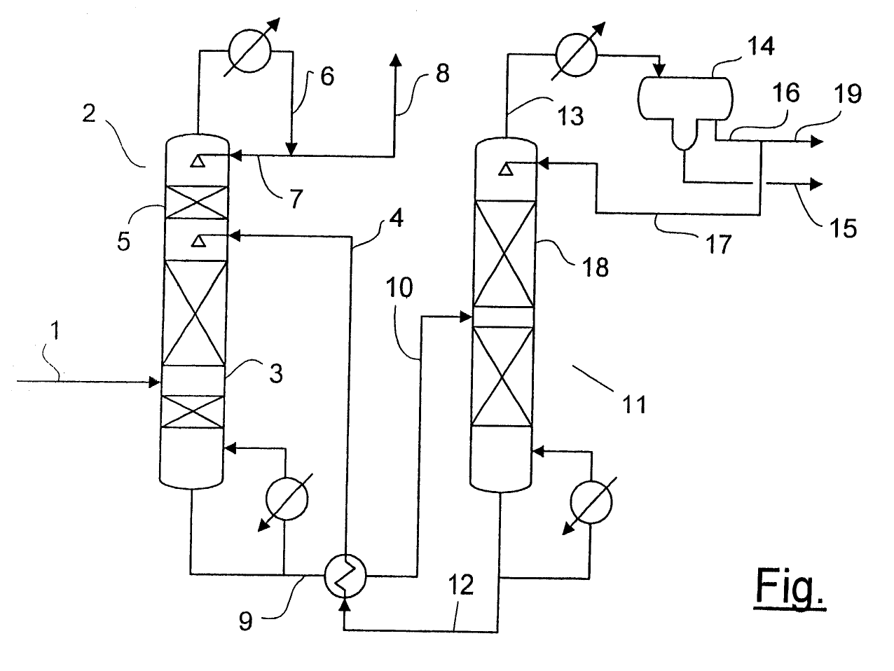

### FIELD OF THE INVENTION

The invention relates to the removal of organic components from an alpha-olefinic crude product. In particular the invention relates to a process for the removal of organic interfering components from an alpha olefin crude product obtained in a Fischer-Tropsch synthesis plant by extractive distillation with a scrubbing agent containing N-Methyl-2-Pyrrolidon (NMP), wherein the NMP is loaded with the oxygenates, is then regenerated and subsequently reused in the extractive distillation.

### BACKGROUND TO THE INVENTION

In coal liquefaction using the Fisher-Tropsch process, a large number of widely varying hydrocarbon compounds are formed. In order to derive the largest economic use from the synthesis process it is necessary to separate the particularly valuable compounds from the profuse mixture and further process these valuable compounds to a required purity.

In recent years a process was developed which permitted the recovery of higher alkenes with a double bond between the first and the second carbon atom, so-called alpha olefins, in particular of pentene and hexene, from such raw materials. The process is known from a lecture delivered at the ACHEMA Congress 1994 from the series “New Processes in Chemical Technology” (C. L. Render, Z. Denga “SASOL alpha olefins”).

Since a large number of other compounds of various types boil in the immediate vicinity of the boiling point of the desired alpha olefins, of which some even produce azeotropic mixtures with the desired alpha olefins, the distillative separation of interfering contaminants is indeed an important process step but it is not possible to attain the desired purity of the alpha olefin product solely by distillation at reasonable expenditure. For that reason, in the known process, components which differ from the alpha olefins to be recovered by virtue of their polarities are separated in a further process step.

These components are mainly paraffins, oxygenates, cyclic olefins and dienes. They are separated from the alpha olefin by extractive distillation.

Although not mentioned in the published concise version of the lecture, an NMP/water mixture having a water content of several weight percent is used as a scrubbing agent in the extractive distillation. Accordingly, this mixture has a boiling point lower than that of pure NMP and permits the use of lower temperatures during regeneration. Since water is a very polar liquid, an increased selectivity was expected in the separation due to the water addition. It is a disadvantage that only a water-saturated olefin product is recovered which, in order to be dried, requires a distillation column and corresponding separation effort. In addition, aqueous NMP—as distinct from the dry form—has a strong foaming tendency.

A need exists for a process by means of which at least some of the above disadvantages are avoided.

### SUMMARY OF THE INVENTION

The invention provides a process for the recovery of at least a fraction of one or more organic interfering components from an alpha olefin crude product recovered in a Fischer-Tropsch synthesis plant by extractive distillation using a scrubbing agent containing N-Methyl-2-Pyrollidon (NMP), wherein the NMP is loaded with the organic interfering components, is then regenerated and subsequently reused in the extractive distillation, wherein the alpha olefin crude product besides the organic interfering components contains water, optionally to saturation point, and the scrubbing agent during regenerating is freed of water to a maximum of 1000 ppm by weight, generally to a maximum of 100 ppm, and typically to a maximum of 10 ppm.

The alpha olefin crude product may predominantly contain 1-hexene.

The alpha olefin crude product may predominantly contain 1-pentene.

An evaluation of relatively recent equilibrium measurements showed surprisingly that the water content has a minor influence only on the selectivity of NMP, so that it does not pay to put up with the disadvantages connected with a higher water content. A lowering of the temperature required for the regeneration can be attained—if this is at all necessary—by a reduction of the regeneration pressure. Although the crude olefin product fed to the extractive distillation in the process according to the invention may be saturated with water, due to the hygroscopic properties of the nearly dry NMP, an olefin product is recovered which contains only a few ppm by weight of water and need not be dried in the majority of cases.

This is in contrast to the process according to the state of the art in which the feed stream of the extractive distillation is inter alia also dried by conventional upstream distillation stages, even though the product during the extractive distillation with the NMP/water mixture is remoisturised, a matter which is disadvantageous from an apparatus and energy point of view.

It is believed to be an advantage of the process according to the invention that the boiling temperature of the loaded scrubbing agent is now almost entirely determined by the hydrocarbon content and not to a major extent by the water content. This temperature may therefore be employed for regulating the running of a plant with the process according to the invention whereby regulating is simplified. In an advantageous embodiment of the process according to the invention, the alpha olefin crude product contains predominantly 1-hexane or 1-pentene.

### DESCRIPTION OF THE DRAWINGS

The invention will now be described, by way of non-limiting example only, with reference to the accompanying diagram.

In the diagram,

the FIGURE shows schematically the removal of cyclic olefins and dienes from a 1-hexene crude product broadly in accordance with the invention.

The crude product 1 is derived from a plant for Fischer-Tropsch synthesis, and contains interfering components which differ by their polarity from 1-hexene, is water saturated and is subjected to an extractive distillation.

As regards the interfering components, these are essentially cyclic olefins and dienes and possibly other hydrocarbons. The 1-hexene crude product 1 is fed into a lower section 3 of an absorber 2 indirectly heated with steam. In addition, a solvent stream 4 including NMP and having a water content of 1 ppm by mass is introduced in which the interfering components are enriched.

Steam rising from the lower section 3 of the absorber 2 is freed in an upper backwashing section 5 of solvent traces. For that purpose a nearly pure 1-hexene is withdrawn in gas form overhead from the absorber 2 and is liquefied. Of this liquid 6 a partial stream 7 is used as reflux in the upper section 5 of the absorber and a further partial stream 8 is recovered as 1-hexene product having a moisture content below 2 ppm by weight and is passed to further use.

From the sump of the absorber 2 a scrubbing agent stream loaded with the interfering components and with water is withdrawn and is fed, preheated at 10 to a regenerating column 11 indirectly heated with steam.

From the sump of the regenerating column 11 regenerated scrubbing agent 12 having a water content of 1 ppm by weight is withdrawn, cooled in counter-current to the reloaded scrubbing agent stream 9 which is heated thereby and is fed to the absorber 2 as the scrubbing agent stream 4.

At the head of the regenerating column 11 a gas flow 13 is withdrawn, condensed and separated by decantation 14 into an aqueous liquid 15 and a liquid 16 containing the interfering components.

A partial stream 17 from the liquid 16 is used as reflux liquor in an upper section 18 of the regenerating column 11 and another portion 19 of the liquid 16 is recovered as by-product stream and forwarded to further use.

What is claimed is:

1. A process for the recovery of at least a fraction of one or more organic interfering components from a Fischer-Tropsch derived alpha olefin crude product containing the organic interfering components and water, the process comprising the steps of:
subjecting the alpha olefin crude product to an extractive distillation using a scrubbing agent containing N-methyl-2-pyrrolidone, whereby the N-methyl-2-pyrrolidone is loaded with at least a fraction of one or more of the organic interfering components;
regenerating the scrubbing agent, such that a regenerated scrubbing agent having a water content of a maximum of 1000 ppm by weight is obtained; and
reusing the regenerated scrubbing agent in the extractive distillation of the alpha olefin crude product.

2. The process of claim 1, wherein the alpha olefin crude product contains water to a saturation point.

3. The process of claim 1, wherein the water content of the regenerated scrubbing agent is a maximum of 100 ppm by weight.

4. The process of claim 2, wherein the water content of the regenerated scrubbing agent is a maximum of 100 ppm by weight.

5. The process of claim 1, wherein the water content of the regenerated scrubbing agent is a maximum of 10 ppm by weight

6. The process of claim 2, wherein the water content of the regenerated scrubbing agent is a maximum of 10 ppm by weight.

7. The process of claim 1, wherein the alpha olefin crude product predominantly contains 1-hexene.

8. The process of claim 2, wherein the alpha olefin crude product predominantly contains 1-hexene.

9. The process of claim 3, wherein the alpha olefin crude product predominantly contains 1-hexene.

10. The process of claim 4, wherein the alpha olefin crude product predominantly contains 1-hexene.

11. The process of claim 5, wherein the alpha olefin crude product predominantly contains 1-hexene.

12. The process of claim 6, wherein the alpha olefin crude product predominantly contains 1-hexene.

13. The process of claim 1, wherein the alpha olefin crude product predominantly contains 1-pentene.

14. The process of claim 2, wherein the alpha olefin crude product predominantly contains 1-pentene.

15. The process of claim 3, wherein the alpha olefin crude product predominantly contains 1-pentene.

16. The process of claim 4, wherein the alpha olefin crude product predominantly contains 1-pentene.

17. The process of claim 5, wherein the alpha olefin crude product predominantly contains 1-pentene.

18. The process of claim 6, wherein the alpha olefin crude product predominantly contains 1-pentene.
